(19) United States
(12) Patent Application Publication
(10) Pub. No.: US 2002/0090359 A1
(43) Pub. Date: Jul. 11, 2002

(54) Transforming growth factor regulation

(76) Inventors: Anthony E. Bolton, Derbyshire (GB); Arkady Mandel, North York (CA); Daniel Nathan Sauder, Baltimore, MD (US)

Correspondence Address: Gerald F. Swiss, BURNS, DOANE, SWECKER & MATHIS, L.L.P., P.O. Box 1404, Alexandria, VA, 22313-1404, US

(21) Appl. No.: 10/002,630

(22) Filed: Dec. 5, 2001

(30) Foreign Application Priority Data
Dec. 5, 2000 (CA) 2,327,630

(51) Int. Cl. A61K 45/00; C12N 5/08

(52) U.S. Cl. 424/93.7; 435/372

(57) ABSTRACT

The present invention relates to a process of increasing the expression of TGF-β₁ by cells in a mammalian patient, comprising administering to the patient an effective amount of stressed mammalian blood cells, said stressed cells having been extracorporeally subjected to at least one stressor selected from oxidative stress and ultraviolet radiation. The process of the present invention shows potential in the treatment of ulcers in mammalian patients.

### CROSS-REFERENCE TO RELATED APPLICATIONS

This application claims priority from Canadian patent application serial number 2,327,630, filed Dec. 5, 2000, the disclosure of which is incorporated herein by reference.

### FIELD OF THE INVENTION

This invention relates to the treatment of biological cells and immune system modulation. More specifically, it relates to treating cells of the mammalian immune system to alter the cytokine profiles of certain types of constituent cells, and therapeutic applications of such treatments.

### BACKGROUND OF THE INVENTION

Transforming growth factor β_{1 (TGF-β}₁), is a cytokine secreted by various mammalian cells, including macrophages, dendritic cells and tissue cells. It appears to play a significant role in the operation of the immune system, by interaction with other component cells thereof after its secretion. The mammalian immune system comprises lymphocytes (one type of white blood cell), the major components of which are B cells, which mature within the bone marrow, and T cells, which migrate from the bone marrow to mature in the thymus gland. B cells react to antigens to proliferate and differentiate into memory B cells and effector B cells which generate and express antibodies specific to the antigen, thereby removing the antigen from the host. T cells have T cell receptors which recognize antigen associated with MHC molecules on a cell, and as a result of this recognition differentiate into memory T cells and various types of effector T cells. The T cell population is made up of T-helper (T_{H}) cells and T-cytotoxic (T_{c}) cells, distinguished from one another by the presence of surface membrane glycoprotein CD4 on T_{H} cells and surface membrane glycoprotein CD8 on T_{c} cells. Activation of a T_{H} cell can cause it to secrete various growth factors (cytokines). Different types of T_{H} cells secrete different cytokines. Cytokines, including TGF-β₁ play key roles in the immune response, including autoimmune responses, often by interacting with other cells to stimulate them into greater production of other cytokines or, conversely, to downregulate them to produce lesser amounts of other cytokines. They can also affect the differentiation and proliferation of cells such as T-cells, to change the population distribution of the various types of T-cells.

While the precise mechanism of action of TGF-β₁ is not fully understood, it is known that TGF-β₁ has various effects on the operation of the immune system. It appears to promote a switch in T_{H} cell type, from T_{H} to T_{H}2, a switch which has benefits in alleviating or hindering the development of autoimmune diseases. It appears to have a role in angiogenesis, suggesting that its presence will have beneficial effects on rates of ulcer healing in the mammalian body.

A process or a medication which would promote the expression of the cytokine TGF-β₁ in a mammalian body would accordingly offer significant benefits to mammalian patients suffering from one or more of a variety of different disorders. It also promotes the healing of ulcers, for example venous ulcers, diabetic ulcers, gastric ulcers, duodenal ulcers, decubitis ulcers, etc. (Danno et.al., “Photodermatol Photoimmunol Photomed Dec. 17, 2001 (6):261-5; Zhou, L J, et.al., Br J Dermatol 2000 September;143(3):506-12;). prolonged pressure and are common in situations were the patient remains in a fixed position for prolonged periods (e.g., long term bed confinement).

In particular, decubitus ulcer formation in nutrient deprived surface skin areas is facilitated by skin irritation due to moisture, friction, and shearing forces. Typically, decubitus ulcer formation is preceded by reddening of nutrient deprived skin which, with continued irritation, develops into the bedsore (i.e., a skin ulcer).

Diabetic ulcers are formed by deprivation of nutrients to the surface skin as a result of the diabetic condition including neuropathy, poor circulation in the patient, etc. In particular, diabetic ulcer formation in nutrient deprived surface skin areas is facilitated by skin irritation due to moisture, friction, and shearing forces. Typically, diabetic ulcer formation is preceded by reddening of nutrient deprived skin which, with continued irritation, develops into a skin ulcer.

Accordingly, it is an object of the present invention to provide a process whereby the expression of the cytokine TGF-β₁ in a mammalian body may be promoted and increased.

It is a further object to provide a composition of matter for administration to a mammalian patient for promoting expression of the cytokine TGF-β₁ in the patient's body.

It is a further object of the present invention to provide a process and composition useful in treating and accelerating the healing of ulcers in a mammalian patient.

### SUMMARY OF THE INVENTION

The present invention provides a process whereby expression of the cytokine TGF-β₁ is promoted in a mammalian patient body. The process involves introducing blood cells into the patient which cells have been extracorporeally stressed by subjecting the blood cells to an oxidative stress and/or ultraviolet radiation. When these stressed blood cells are introduced to the patient, they appear to have the effect of promoting the expression of TGF-β₁, either by activating and upregulating one of the types of mammalian cells which naturally express it, or by increasing the relative population of such cells, or both. Whatever the precise mechanism of action, the result is a significant and measurable increase in TGF-β₁ levels in the patient's system. Accordingly, the process of the invention is useful for the medical treatment of patients suffering from, prone to, or at risk of contracting a disorder associated with insufficient amounts of TGF-β₁. It also provides a process of accelerating the healing of wounds. Since increased levels of TGF-β₁ are found in the dermis of human patients who have been given treatments according to the invention, the process is particularly indicated for the healing of skin ulcers, such as decubitis ulcers, diabetic ulcers and the like.

Thus according to the present invention, there is a provided a process of increasing the expression of TGF-β₁ by cells in a mammalian patient, which comprises administering to the patient an effective amount of stressed mammalian blood cells, said stressed cells having been extracorporeally subjected to at least one stressor selected from oxidative stress and ultraviolet radiation.

From another aspect, the present invention provides a composition of matter for administration to a mammalian patient to raise the levels of expressed TGF-β₁ in the patient's system, wherein said composition comprises stressed blood cells from the patient, the cells having been stressed by subjecting them extracorporeally to at least one stressor selected from oxidative stress and ultraviolet light.

### BRIEF REFERENCE TO THE DRAWINGS

FIGS. 1 and 2 of the accompanying drawings are graphical presentations of results obtained according to the experiment of Example 1, described below.

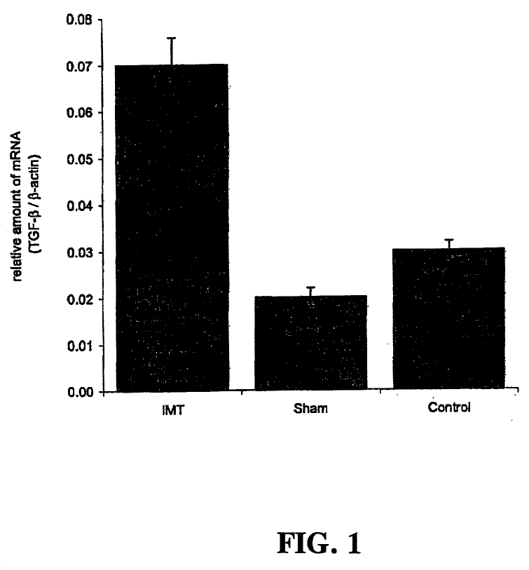

### DESCRIPTION OF THE PREFERRED EMBODIMENTS

The source of the stressed blood cells for use in the present invention is preferably the patient's own blood, i.e. an aliquot of autologous blood.

The terms “aliquot”, “aliquot of blood” or similar terms used herein include whole blood; separated cellular fractions of the blood, including platelets; separated non-cellular fractions of the blood, including plasma; plasma components; and combinations thereof. Preferably, in human patients, the volume of the aliquot is up to about 400 ml, preferably from about 0.1 to about 100 ml, more preferably from about 1 to about 15 ml, even more preferably from about 8 to about 12 ml, and most preferably about 10 ml. The effect of the stressor or the combination of stressors is to modify the blood, and/or the cellular or non-cellular fractions thereof, contained in the aliquot. The modified aliquot is then re-introduced into the patient's body by any suitable method, most preferably intramuscular injection, but also including subcutaneous injection, intraperitoneal injection, intra-arterial injection, intravenous injection and oral administration, following which it causes an increase in the expression of TGF-β₁ by the patient.

According to a preferred process of the present invention, an aliquot of blood is extracted from a mammalian subject, preferably a human, and the aliquot of blood is treated ex vivo, simultaneously or sequentially, with the aforementioned stressors. The blood is then injected back into the same subject. Preferably a combination of both of the aforementioned stressors is used.

Preferably, the aliquot of blood is further subjected to mechanical stress. Such mechanical stress is suitably that applied to the aliquot of blood by extraction of the blood aliquot through a conventional blood extraction needle, or a substantially equivalent mechanical stress applied shortly before the other chosen stressors are applied to the blood aliquot. This mechanical stress may be supplemented by the mechanical stress exerted on the blood aliquot by bubbling gases through it, such as ozone/oxygen mixtures, as described below. Optionally also, a temperature stressor may be applied to the blood aliquot, simultaneously or sequentially with the other stressors, i.e. a temperature at, above or below body temperature.

The optionally applied temperature stressor either warms the aliquot being treated to a temperature above normal body temperature or cools the aliquot below normal body temperature. The temperature is selected so that the temperature stressor does not cause excessive hemolysis in the blood contained in the aliquot and so that, when the treated aliquot is injected into a subject, the desired effect will be achieved, without development of significant adverse side effects. Preferably, the temperature stressor is applied so that the temperature of all or a part of the aliquot is up to about 55° C., and more preferably in the range of from about −5° C. to about 55° C.

In some preferred embodiments of the invention, the temperature of the aliquot is raised above normal body temperature, such that the mean temperature of the aliquot does not exceed a temperature of about 55° C., more preferably from about 40° C to about 50° C, even more preferably from about 40° C. to about 44° C., and most preferably about 42.5±1° C.

In other preferred embodiments, the aliquot is cooled below normal body temperature such that the mean temperature of the aliquot is within the range of from about 4° C. to about 36.5° C., more preferably from about 10° C. to about 30° C., and even more preferably from about 15° C. to about 25° C.

The oxidative environment stressor can be the application to the aliquot of solid, liquid or gaseous oxidizing agents. Preferably, it involves exposing the aliquot to a mixture of medical grade oxygen and ozone gas, most preferably by applying to the aliquot medical grade oxygen gas having ozone as a component therein. The ozone content of the gas stream and the flow rate of the gas stream are preferably selected such that the amount of ozone introduced to the blood aliquot, either on its own or in combination with one of the other stressors, does not give rise to excessive levels of cell damage, and so that, when the treated aliquot is injected into a subject, the desired effect will be achieved, without development of significant adverse side effects. Suitably, the gas stream has an ozone content of up to about 300 μg/ml, preferably up to about 100 μg/ml, more preferably up to about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 litres/min, preferably up to about 0.5 litres/min, more preferably up to about 0.4 litres/min, even more preferably up to about 0.33 litres/min, and most preferably about 0.24±0.024 litres/min. The lower limit of the flow rate of the gas stream is preferably not lower than 0.01 litres/min, more preferably not lower than 0.1 litres/min, and even more preferably not lower than 0.2 litres/min, all rates at STP (0° C. and 1 atmosphere pressure).

The ultraviolet light stressor is suitably applied by irradiating the aliquot under treatment from a source of UV light, i.e. electromagnetic radiation of wavelength from about 180 nm to about 400 nm. Preferred UV sources are UV lamps emitting UV-C band wavelengths, i.e. at wavelengths shorter than about 280 nm. Ultraviolet light corresponding to standard UV-A (i.e., wavelengths from about 315 to about 400 nm) and UV-B (i.e., wavelengths from about 280 to about 315) sources can also be used. As in the case of the oxidative stressor, the UV dose should be selected, on its own or in combination with the other chosen stressor(s), so that excessive amounts of cell damage do not occur, and so that, when the treated aliquot is injected into a subject, the desired effect will be achieved. For example, an appropriate dosage of such UV light, applied simultaneously with the aforementioned temperature and oxidative environment stressor, can be obtained from lamps with a power output of from about 10 to about 30 watts, arranged to surround the sample container holding the aliquot, each lamp providing an intensity, at a distance of 16 mm, of from about 5 to about 20 mW/cm². Up to eight such lamps surrounding the sample bottle, with a combined output at 253.7 nm of 10 to 30 watts, operated at an intensity to deliver a total UV light energy at the surface of the blood of from about 0.025 to about 10 joules/cm², and preferably from about 0.1 to about 3.0 joules/cm², may advantageously be used. Such a treatment provides a modified blood aliquot which is ready for injection into the subject.

It is preferred to subject the aliquot to the oxidative environment stressor, the UV light stressor and the temperature stressor simultaneously, following the subjection of the aliquot to the mechanical stress, e.g. by extraction of the blood from the patient. Thus, the aliquot may be maintained at a predetermined temperature above or below body temperature while the oxygen/ozone gas mixture is applied thereto and while it is irradiated with ultraviolet light.

The time for which the aliquot is subjected to the stressors is normally within the time range of from about 0.5 minutes up to about 60 minutes. The time depends to some extent upon the chosen combination of stressors. When an UV light is used, the intensity of the UV light may affect the preferred time. The chosen temperature level may also affect the preferred time. When an oxidative environment in the form of a gaseous mixture of oxygen and ozone applied to the aliquot is chosen as one of the two stressors, the concentration of the oxidizing agent and the rate at which it is supplied to the aliquot may affect the preferred temperature. Some experimentation to establish optimum times may be necessary on the part of the operator, once the other stressor levels have been set. Under most stressor conditions, preferred times will be in the approximate range of from about 2 to about 12 minutes, more preferably from about 2 to about 5 minutes, most preferably about 3 minutes. The starting blood temperature, and the rate at which it can be warmed or cooled to a predetermined temperature, tends to vary from subject to subject. Warming is suitably by use of one or more infrared lamps placed adjacent to the aliquot container. Other methods of warming can also be adopted.

As noted, it is preferred to subject the aliquot of blood to a mechanical stressor, as well as the chosen stressor(s) discussed above. Extraction of the blood aliquot from the patient through an injection needle constitutes the most convenient way of obtaining the aliquot for further extracorporeal treatment, and this extraction procedure imparts a suitable mechanical stress to the blood aliquot. The mechanical stressor may be supplemented by subsequent processing, for example the additional mechanical shear stress caused by bubbling as the oxidative stressor is applied.

In the practice of the preferred process of the present invention, the blood aliquot may be treated with the heat, UV light and oxidative environment stressors using an apparatus of the type described in aforementioned U.S. Pat. No. 4,968,483 to Müller et al. The aliquot is placed in a suitable, sterile container, which is fitted into the machine. A UV-permeable container is used and the UV lamps are switched on for a fixed period before the other stressor(s) is applied, to allow the output of the UV lamps to stabilize. When a temperature stressor is used in combination with UV light stressors, the UV lamps are typically on while the temperature of the aliquot is adjusted to the predetermined value, e.g. 42.5±1° C. Four UV lamps are suitably used by placing around the aliquot containing container.

In the preferred method of the invention, a mammalian patient standing to benefit from an increased expression of TGF-β₁ is given one or more courses of treatments, each course of treatment comprising the administration to the mammalian subject of one or more (e.g., one to six or one to twelve) aliquots of mammalian blood modified as discussed above.

For optimum effectiveness of the treatment, it is preferred that no more than one aliquot of modified blood be administered to the subject per day in one or more injection sites, and that the maximum rest period between any two consecutive aliquot administrations during the course of treatment should be no greater than about 21 days. As used herein, the term “rest period” is defined as the number of days between consecutive aliquots or consecutive courses of treatment on which no aliquots of modified blood are administered to the subject.

Therefore, except where aliquots are administered to the subject on consecutive days, a rest period of from 1 to 21 days is provided between any two aliquots during the course of treatment. Moreover, at least one of the rest periods during the course of treatment preferably has a length of about 3 to 15 days.

Although it may be sufficient to administer only one course of treatment as described above to the subject, it may be preferred in some circumstances to administer more than one course of treatment, or to follow the above-described course of treatment by periodic “booster” treatments, if necessary, to maintain the desired effects of the present invention. For example, it may be preferred to administer booster treatments at intervals of 1 to 4 months following the initial course of treatment, or to administer a second course of treatment to the subject following a rest period of several weeks or months.

The invention is further illustrated and described below with reference to Example 1, comprising animal studies conducted in an approved manner, and Example 2, a clinical trial on human patients. The examples are offered for purposes of illustrating the invention and should not be construed as a limitation.

### EXAMPLE 1

Whole blood was obtained from Balb/c mice, by cardiac puncture through an injection needle, and treated with an anti-coagulant. An aliquot of this anti-coagulated blood was subjected to the process of a preferred embodiment of the invention, to obtain treated blood. The remainder was left untreated, for use in control experiments. Since the Balb/c mice used were genetically identical, the administration of the treated blood to others of the group is equivalent to administration of autologous blood.

To obtain treated blood, the selected aliquot, in a sterile, UV-transmissive container, was treated simultaneously with a gaseous oxygen/ozone mixture and ultraviolet light at elevated temperature using an apparatus as generally described in aforementioned U.S. Pat. No. 4,968,483 Müller et.al. Specifically, 12 ml of citrated blood was transferred to a sterile, low density polyethylene vessel (more specifically, a Vasogen VC7002 Blood Container) for ex vivo treatment with stressors according to the invention. Using an apparatus as described in the aforementioned Muiller et al. patent (more specifically, a Vasogen VC7001 apparatus), the blood was heated to 42.5±1° C. and at that temperature irradiated with UV light at a wavelength of 253.7 nm, while oxygen/ozone gas mixture was bubbled through the blood to provide the oxidative environment and to facilitate exposure of the blood to UV. The constitution of the gas mixture was 14.5±1.0 μg ozone/ml, with the remainder of the mixture comprising medical grade oxygen. The gas mixture was bubbled through the aliquot at a rate of 240±24 ml/min for a period of 3 minutes.

There were 4 groups of Balb/c mice. The first, control group A-1 received no treatment. The second, control group B-1, was treated with 50 μl of physiological saline. The third, control group C-1;, was sham treated with 50 μl of blood which had been extracted, but not treated with the additional stressors. The fourth, test group D-1, was treated with 50 μl of blood subjected to stressors as described above. Treatments, each involving injection of 50 μl of the respective liquid into the gluteal muscle, started on day 1, and were repeated every day for a total of 6 days.

The experiment was run in parallel to the test for contact hypersensitivity resistance in the mice, as described in applicants co-pending international patent application PCT/CA00/00433 incorporated herein by reference, so that the various groups had been pre-sensitized with dinitrofluorobenzene (DNFB) and were subsequently challenged on one ear, 24 hours after the last injection, with DNFB as described therein, but this is not a factor in the tests demonstrating the present invention.

Each of the animals was sacrificed and the lymph nodes draining the ear that was challenged with DNFB were collected. The expression of the mRNA of the cytokine TGF-β₁ in the lymph tissue so obtained was analyzed using known RT-PCR techniques, essentially following the procedures described in Kondo et.al., J. Immunology, Vol. 157:4822, 1996. The PCR products were determined by scanning of photonegatives using a laser densitometer, and the densitometric value of the TGF-β₁ was normalized to that of the housekeeping gene β-actin. The analyses indicated that animals which had received a course of injection of blood subjected to stressors as described had significantly increased concentrations of TGF-β₁ in the lymph node, as compared with controls and sham treated animals. The analyses were repeated three times, and the accompanying Figure illustrates the mean of these results.

### EXAMPLE 2

A total of 20 human patients having moderate to severe psoriasis were randomized into a double blind, placebo controlled clinical trial. Two groups of 10 patients received 2 injections per week intramuscularly, into the gluteal muscle, of treated blood or saline, over a 3 week period. The therapy involved the collection of 10 ml of the patient's venous blood into 2 ml sodium citrate. The blood was transferred to a sterile disposable low-density polyethylene vessel for ex vivo treatment as described in Example 1. Prior to muscular injection, 1 ml of Novocain was injected into the gluteal muscle as a local anesthetic.

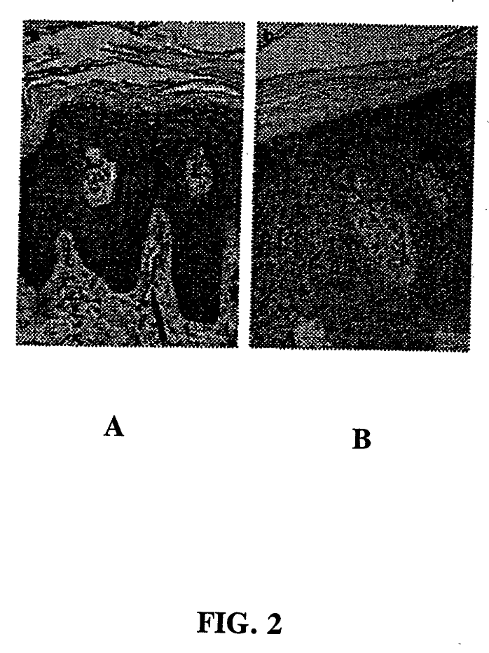

Skin biopsies were taken at the end of the treatment, fixed in formalin and embedded in paraffin. Histological examination of skin biopsies of patients who had undergone treatment according to the invention was undertaken, by immunohistochemistry using a monoclonal antibody to TGF-β₁. Increased production of TGF-β₁ in the dermis of patients treated according to the invention was seen in slides of tissue (FIG. 2a, microphotograph of the biopsied human skin sample after treatment to visualize TGF-β₁ ) based on increased density of staining in TGFβ₁ producing cells, compared to patients treated with saline (FIG. 2b). This result is indicative of the use of the process of the invention to upregulate TGF-β₁ and therefore in treating ulcers of the skin.

All references cited above are herein incorporated by reference in their entirety.

1. A process of increasing the expression of TGF-βₗ from cells in a mammalian patient, which comprises administering to the patient an effective amount of stressed mammalian blood cells, said stressed cells having been extracorporeally subjected to at least one stressor selected from oxidative stress and ultraviolet radiation, and thereby alleviating in the patient the symptoms of a disorder associated with TGF-β₁ deficiency.

2. A process of treating ulcers in a mammalian patient to accelerate the healing thereof, which comprises administering to an ulcer—suffering patient an effective amount of stressed mammalian blood cells, said stressed cells having been extracorporeally subjected to at least one stressor selected from oxidative stress and ultraviolet radiation, and thereby accelerating the healing of the patient's ulcer.

3. The process of claim 2 wherein the stressed blood cells originate from the same patient.

4. The process of claim 3 wherein the stressed cells have been subjected to both oxidative stress and ultraviolet radiation simultaneously.

5. The process of claim 4 wherein the stressed mammalian blood cells have been additionally extracorporeally subjected to heat stress simultaneously with the subjection to both oxidative stress and ultraviolet radiation.

6. The process of claim 5 wherein the oxidative conditions comprise bubbling a gaseous mixture of medical grade oxygen and ozone through the blood, for a period of from about 0.5 minutes-60 minutes.

7. The process of claim 6 wherein the gaseous mixture has an ozone content of from about 0.1 to about 100 μg/ml.

8. The process of claim 7 wherein the UV stressor is UV-C radiation.

9. The process of claim 8 wherein the temperature stressor is a temperature in the range of from about 40 to about 55° C.

10. The process of claim 9 wherein the stressed mammalian blood cells comprise a volume of whole blood of from about 0.1 to about 400 mls.

11. A process of inhibiting ulcer formation in a mammalian patient at risk of said ulcer formation which comprises administering to said patient an effective amount of stressed mammalian blood cells, said stressed cells having been extracorporeally subjected to at least one stressor selected from oxidative stress and ultraviolet radiation, and thereby accelerating the healing of the patient's ulcer.

12. The process of claim 11, wherein the stressed blood cells originate from the same patient.

13. The process of claim 11, wherein the stressed blood cells have been subjected to both oxidative stress and ultraviolet radiation simultaneously.

14. The process of claim 11, wherein the stressed mammalian blood cells have been additionally extracorporeally subjected to heat stress simultaneously with the subjection of both oxidative stress and ultraviolet radiation.

15. The process of claim 11, wherein the oxidative conditions comprise bubbling a gaseous mixture of medical grade oxygen and ozone through the blood, for a period of from about 0.5 minutes to about 60 minutes.

16. The process of claim 15, wherein the gaseous mixture has an ozone content of from about 0.1 to about 100 μg/ml.

17. The process of claim 11, wherein the UV stressor is UV-C radiation.

18. The process of claim 14, wherein the temperature stressor is a temperature in the range of from about 40 to about 55° C.

19. The process of claim 11, wherein the stressed mammalian blood cells comprise a volume of whole blood of from about 0.1 to about 400 mls.
